(19) United States
(12) Patent Application Publication
(10) Pub. No.: US 2005/0070817 A1
(43) Pub. Date: Mar. 31, 2005

(54) Lavage assist device

(76) Inventor: Richard L. Mueller JR., Jackson, WY (US)

Correspondence Address: OLSON & HIERL, LTD., 36th Floor, 20 North Wacker Drive, Chicago, IL, 60606, US

(21) Appl. No.: 10/675,294

(22) Filed: Sep. 30, 2003

(51) Int. Cl. A61B 10/00; A41C 3/00

(52) U.S. Cl. 600/563; 450/37

(57) ABSTRACT

A harness and method for reducing systemic losses in ductal lavage procedures is provided comprising a base member having a first surface and a second surface and a through aperture sized to receive a human breast therethrough. The through aperture includes a breast stabilizer that is suitable for compressing at least a portion of the breast near or proximate the chest. The compression of the breast radially is sufficient to act as a moderate tourniquet on the breast to reduce systemic losses.

### TECHNICAL FIELD OF THE INVENTION

This invention relates generally to ductal lavage assist devices and kits for use in clinical procedures performed on a human female breast. More specifically, the invention relates to a harness for stabilizing and positioning a human female breast during clinical diagnostic and surgical procedures on the breast so as to reduce systemic losses of lavage liquid.

### BACKGROUND OF THE INVENTION

Breast cancer is one of the most common forms of cancer in women. A key to treatment is early detection. For example, annual mammograms have been recommended in hopes of early detection of breast cancer. One problem with mammographic imaging is that it can only detect breast cancer once it has taken tangible form as a tumor. All too often, breast cancer is discovered at a stage that is too far advanced, when therapeutic options and survival rates are severely limited. Therefore, more sensitive and reliable methods and devices are needed to detect cancerous, pre-cancerous, and other cancer indicators of the breast at an early stage. Such methods and devices could significantly improve breast cancer survival. While breast cancer is most common among women, in rare instances breast cancer can occur in men.

A vast majority of breast cancers reportedly begin in the lining of mammary ducts. Studies have indicated that fluid within the mammary ducts can contain high levels of breast cancer markers, and that an estimated 80% to 90% of all breast cancers occur within the intraductal epithelium of the mammary glands. The fluid within the breast ducts contains an assemblage and concentration of hormones, growth factors and other potential markers comparable to those secreted by, or acting upon, the surrounding cells of the alveolar-ductal system. Mammary fluid also typically contains cells and solid cellular debris or products that can be used in cytological or immunological assays for breast cancer.

An early method of detecting breast cancer based on analysis of mammary fluid involves analyzing a sample of mammary fluid that was excreted by the mammary ducts without outside intervention. In other words, if a mammary duct is actively discharging a fluid, this fluid is analyzed. Often breasts do not discharge fluid spontaneously, even though breast cancer markers may be present in the mammary fluid.

One such diagnostic procedure based on the analysis of mammary fluid is ductal lavage. This method entails introducing a saline solution or other rinsing liquid as a lavage liquid into the mammary ducts via a small catheter and removing the introduced solution from the mammary ducts by vacuum and massage. The recovered liquid is then subjected to immunological or cytological evaluation for the presence of breast cancer markers. This is a delicate procedure requiring considerable skill on the part of the clinician performing the catheterization. One advantage of such methods is that fluid samples can be retrieved from individual ducts, thus facilitating location of the cancerous area within the breast. One difficulty with such procedures is that only a small portion of the saline introduced is recovered by vacuum due to systemic losses of upwards of 50%. It is common for practitioners to introduce 2-10 cc of the lavage rinse, but to only recover 1-5 cc. The introduction and retrieval process for the lavage liquid can be repeated to gain additional specimen rich samples for cytological evaluation. However, this requires additional patient and assistant time.

The present invention provides a harness-type device and kit for stabilizing and positioning a human breast for lavage diagnostic or surgical procedures with minimal manual intervention to maintain the breast in a suitable position and optimal condition during the procedure. The harness of the present invention also serves to apply circumferential pressure to the base of the human breast to reduce systemic losses of the lavage liquid.

### SUMMARY OF THE INVENTION

A harness for reducing systemic losses of lavage liquid during ductal lavage procedures is provided. The harness also is also suitable for stabilizing and positioning a human breast for lavage diagnostic or surgical procedures with minimal manual intervention to maintain the breast in a suitable position and optimal condition during the procedure.

The harness comprises a base member that defines a trhough aperture and has a first surface and a second surface. The through aperture is sized to receive a human breast therethrough. The through aperture is surrounded by a breast stabilizer that is suitable for compressing at least a portion of the breast near or proximate the chest. The breast stabilizer, in use, is preferably positioned near the base of the breast (region nearest the chest wall) and is configured to exert a compressive force on connective tissue proximate to where the breast adjoins the chest wall and to the base of the breast. The compression of the breast radially is sufficient to act as a moderate tourniquet on the breast to reduce losses of liquids introduced during a ductal lavage procedure, i.e., systemic losses. The compressive force is not such that blood flow is interrupted, however. The harness and breast stabilizer are preferably configured such that they may be utilized without obstructing massage surfaces of the breast.

The harness base member also preferably includes a torso band having a first end portion and a second end portion, and is suitable for enveloping and securement about the chest of a human female patient. The harness base member is preferably formed of an elastomeric material. The torso band is secured about the chest of a human female on which the procedure is to be performed. The breast subject to the procedure extends through the aperture, and the associated breast stabilizer. In order to secure the harness about the patient, the first end portion of the torso band may be engageable with the second end portion of the torso band. For example, the first surface of the first end portion can be engaged with the second surface of the second end portion, such as through use of a hook and loop engagement, e.g., a VELCRO®-type fastener. In a preferred embodiment, the second surface of the second end portion includes a web of entangled fibers, and at least a portion of the first surface includes a plurality of flexible hooks, the plurality of flexible hooks being engageable with the web of entangled fibers. However, any device for removably engaging the first and second end portions may be utilized, such an adhesive, buckle, latch, or the like.

In some embodiments the breast stabilizer is integral, and may also be unitary, with the base member. For example, the breast stabilizer may be comprised of an elastomeric material similar to that of the base member. The through aperture is of a smaller diameter than the base diameter of the patient's breast. The human breast may be passed through the aperture, and due to the elastic nature of the breast stabilizer, the aperture can be stretched or expanded to permit such passage. The breast stabilizer, however, is radially inwardly biased due to rebound properties of the material, and therefore compresses a circumferential region of the base of the breast proximate or near the patient's chest. The elastomeric characteristics of the breast stabilizer may optionally be different from the base member. For example, a more resilient elastomer may be used in the breast stabilizer to create a greater amount of radial compressive force, such as for larger breasted women, whose breast have a greater tendency to sag and roll.

Alternatively, the breast stabilizer may include a manual adjustment mechanism or clamp to create the radial compression, such as an adjustable belt forming a collar or ring or adjustable tabs. The compression on the circumferential region of the base of the breast proximate or near the patient's chest can be adjusted as desired by such a mechanism or clamp.

As another alternative, the harness may include a plurality of apertures being of different diameters to accommodate a variety of breast sizes. Yet another alternative includes an inflatable bladder circumscribing the aperture, which when inflated, increases the radial compression on the breast. It is also possible for the adjustment mechanism to be automatically adjusted. For example, with the inflatable bladder, a pressure sensor in communication with a pump may be included to automatically maintain a predetermined amount of force exerted on the breast.

In order to provide some privacy and comfort for the patient, the harness may optionally include an elongated slit or opening spaced from the through aperture. The elongated slit or opening is suitable for allowing passage of the patient's other breast therethrough. Preferably, the base member would also include a cover over the elongated slit or opening to cover the breast. As such, the harness and torso band may be secured about the patient without having to compress the patient's other breast during the procedure.

In a preferred embodiment, the harness also includes a sanitary liner layer disposed on the base member second surface. The liner layer may be removable so that it can be replaced after each use, if desired. The liner can also be made of a material that wicks moisture, such as perspiration, away from the patient to further accommodate the patient's comfort.

One advantage of the present invention is to provide a device for reducing the degree of systemic losses of ductal lavage solution to increase the yield recovered from a single procedure. The harness exerts sufficient circumferential force on the base of the breast to act as a tourniquet and reduce such losses. Constriction of the base of the breast by the support harness can also counteract breast nipple inversion, which occurs in some patients when in supine position with the breast elevated.

Another benefit of the harness is to provide for stabilization and positioning of the human breast during an operative or clinical diagnostic procedure. The harness provides support to hold the breast in a more optimized operative position by reducing the tendency for breast sag or roll while not obstructing the operative surfaces of the breast and nipple.

The present invention also encompasses kits containing at least one harness of the present invention, as well as ductal lavage related devices, such as catheters, syringes, dilators and massage aids.

### BRIEF DESCRIPTION OF THE DRAWINGS

In the drawings,

FIG. 1 is a perspective schematic view of an embodiment of a harness including a torso band;

FIG. 2 is a perspective schematic view of the harness of FIG. 1 as applied to a patient;

FIG. 3 is an enlarged, fragmentary cross sectional side view of the base member material of the embodiment of FIG. 1;

FIG. 4 is an enlarged partial side view of a harness illustrating a lavage procedure;

FIG. 5 is a partial perspective view of an alternate embodiment of the harness further including a plurality of apertures of different diameters;

FIG. 6 is a perspective view of a harness including a torso band, and the breast stabilizer further including a manual belt adjustment;

FIG. 7 is an enlarged partial perspective view of an alternate embodiment of a harness including a torso band, and the breast stabilizer further including a manual belt adjustment;

FIG. 8 is an enlarged partial perspective view of a harness including a torso band, and the breast stabilizer further including a plurality of slits with adjustable tab enclosures;

FIG. 9 is an enlarged partial bottom perspective view of a harness including a torso band, and the breast stabilizer further including an inflatable bladder;

FIG. 10 is a perspective view of an embodiment of a harness without a torso band;

FIG. 11 is a perspective view of an alternate embodiment of a harness without a torso band; and

FIG. 12 is a perspective schematic view of a kit of the present invention.

### DETAILED DESCRIPTION OF PREFERRED EMBODIMENTS

The invention disclosed herein is susceptible of being embodied in many different forms. Shown in the drawings and described herein below in detail are preferred embodiments of the invention. It is to be understood, however, that the present disclosure is an exemplification of the principles of the invention and does not limit the invention to the illustrated embodiments.

As used herein and in the appended claims, the term “breast nipple” and grammatical variations thereof refers to the entire areola region of the breast including the areola and the protruding portion thereof commonly referred to as the “nipple.”

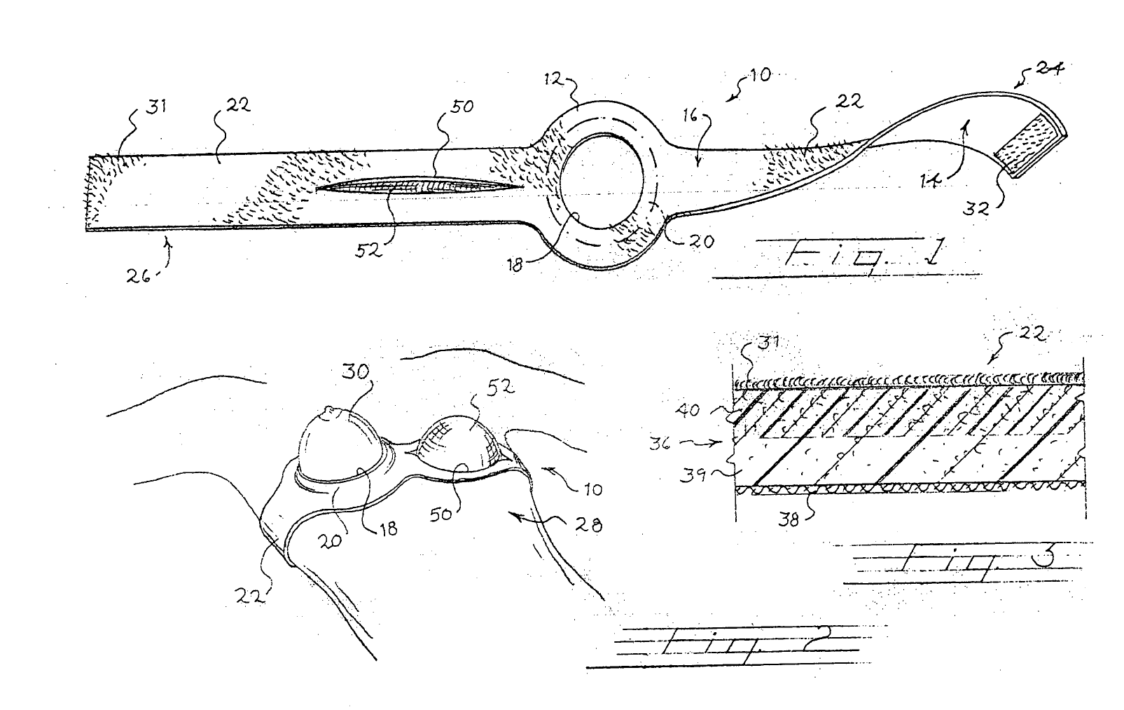

An embodiment of a harness 10 for reducing systemic losses in ductal lavage procedures is shown in FIG. 1. The harness 10 also is suitable for stabilizing and positioning a human breast for lavage diagnostic or surgical procedures with minimal manual intervention to maintain the breast in a suitable position and optimal condition during the procedure.

The harness 10 comprises a base member 12 having a first surface 14 and a second surface 16 and a through aperture 18 sized to receive a human breast therethrough. Located circumferentially about the aperture 18 is provided a breast stabilizer 20. As will be discussed in further detail below, the breast stabilizer 20 is suitable for compressing at least a portion of the base of breast, i.e., near or proximate the chest wall of the patient. In this particular embodiment, the breast stabilizer 20 is an elastic aperture ring that is unitary with the base member. The breast stabilizer 20, in use, surrounds aperture 18 and exerts a compressive force on connective tissue proximate to where the breast adjoins the chest wall and to the base of the breast. The resulting radial compression of the breast is sufficient to act as a moderate tourniquet to reduce losses of liquids introduced during a ductal lavage procedure, i.e., systemic losses, but is not such that blood flow to the breast is interrupted.

Preferably, the harness base member 12 includes a torso band 22 having a first end portion 24 and a second end portion 26, and is suitable for enveloping and securement about the chest of a human female patient as shown in FIG. 2. The torso band 22 is secured about the torso 28 of a human female on which the procedure is to be performed. The breast 30 subject to the procedure extends through the aperture 18, and the associated breast stabilizer 20. In order to secure the harness 10 to the patient, the torso band 22 is wrapped around the patient's torso 28. The first end portion 24 (FIG. 1) of the torso band 22 is engaged with the second end portion 26 (FIG. 1) of the torso band 22. For example, the first surface 14 of the first end portion 24 can be engaged with the second surface 16 of the second end portion 26, such as through use of a hook and loop engagement. In order to provide some privacy and comfort for the patient, the harness may optionally include an elongated slit 50 spaced from the through aperture 18. The elongated slit 50 is suitable to allowing passage of the patient's other breast therethrough. Preferably, the base member 12 also includes a cover 52 over the elongate slit 50 to cover the breast that is not being examined as shown in FIG. 2.

Referring again to FIG. 1, in a preferred embodiment, the second surface 16 of at least the second end portion 26 includes a web of entangled fibers 31, and at least a portion of the first surface 14 includes a plurality of flexible hooks 32, the plurality of flexible hooks 32 being engageable with the web of entangled fibers 31. The entire second surface 16 may include entangled fibers 31 such that many different engagement options are available.

In this embodiment, the breast stabilizer 20 circumscribes the entire aperture and comprises an elastomeric material that is integral, and may also be unitary, with the base member 12. The through aperture 18 is of a smaller diameter than the base diameter 34 of the patient's breast 30. The human breast 30 may be passed through aperture 18, and due to the elastic nature of the breast stabilizer 20, the aperture 18 can be stretched to permit such passage. The breast stabilizer 20, however, is radially inwardly biased due to rebound properties of the material, and therefore compresses the base diameter 34 of the breast 30. Although not shown, the breast stabilizer 20 may also include an internal rib to enhance the localization of the compressive force.

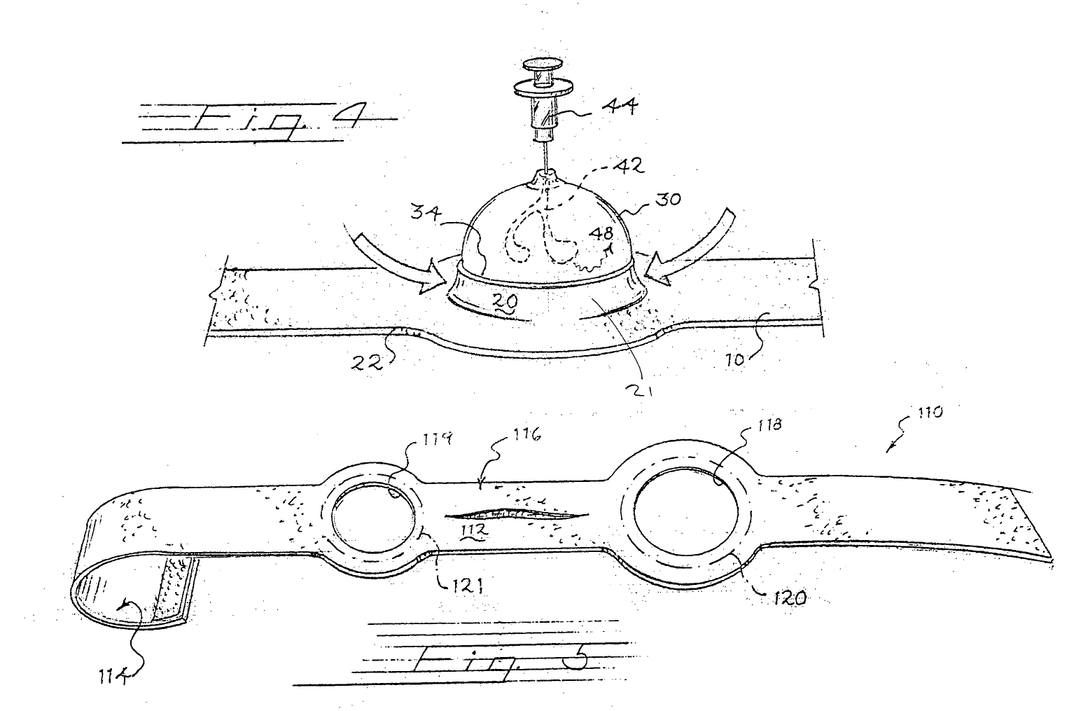

The torso band 22 preferably is made of a strip of fabric, which is preferably a stretchable resilient fabric. A preferred material of construction for the torso band 22 is shown in FIG. 3. The torso band 22 is constructed from a resilient composite fabric, such as a trilaminate fabric 36, which includes a liner layer 38, such as a nylon or polyester web, and the like; an elastomeric, foamed core layer 39, such as a neoprene foam, and the like; and an outer surface layer 40 including a self-supporting web of entangled fibers 31 suitable for engagement with the hook portion of a hook and loop fastener. Such preferred fabrics are commercially available and are commonly used in wet suit manufacture. A particularly preferred fabric is a stretchable trilaminate fabric having a neoprene foam core layer, a nylon or polyester liner layer and a surface layer comprising an unbroken loop (UBL) fabric that is suitable for engaging the hooks of a hook and loop fastener. Preferred fabrics are commercially available from RBX Industries, Inc., Roanoke, Va., under the trade name RUBATEX®, such as RUBATEX R-470-N having a 200 series UBL outer layer, and a nylon or polyester liner layer. The harness 10, including the base member 12 and torso band 22, may all be formed of the same resilient material. For ease of manufacturing, one may cut out the harness from a sheet of material and add the flexible hooks for securement. In such an embodiment, the entire second surface 16 would include the web of fibers 31.

Alternatively, the torso band can be constructed of any convenient fabric, including, for example, cotton, acrylic, nylon, and the like, and the ends of the band can be fitted with complementary portions of at least one suitable fastener, such as a hook and eye fastener, a hook and loop fastener, such as a VELCRO® brand hook and loop fastener, a snap button fastener, a side release plastic buckle, a center release plastic buckle, a ladder lock buckle, a press buckle, and the like.

In clinical use, the harness 10 can be made inexpensively enough to be used as a disposable. Configuring the product as a disposable minimizes clinic cleaning issues as patient sweat may contaminate the harness 10. In a disposable configuration the harness 10 can also be offered as a small, medium and large size more easily. To that end, liner layer 38 can be comprised of a hypoallergenic material that also wicks perspiration away from the patient, while also trapping said moisture away from the base member 12. Alternatively a disposable liner or drape can utilized that is removable and replaceable as the liner layer 38. This drape may extend the need for washings or multiple drapes can be provided in a kit with a single 5-10 use harness.

Referring to FIG. 4, the reduction of systemic loss of liquid though application of the present invention is described. As shown, breast 30 is passed through aperture 18. Breast stabilizer 20 exerts a compressive force on the base of breast 30. A lavage solution is introduced into a mammary duct 42 by syringe 44. Without the harness 10, a substantial portion of the lavage solution is lost as the body tissues transport the lavage solution from the mammary duct. When the harness 10 is used to position and stabilize a breast 30 for a diagnostic procedure, such as for a ductal lavage, the torso band 22 and the breast stabilizer 20 having the configuration of elastomeric band 21 provide gentle tolerable compressive pressure to the chest wall surrounding the breast and gentle tolerable circumferential clamping at the base 34 of the breast 30. These two gentle clamping forces in combination or separately reduce the tendency for lavage fluids to readily leave the ductal area (systemic loss), and therefore become unrecoverable by vacuum and massage techniques. The compression of the base 34 of breast 30 by breast stabilizer 20 reduces the amount of lavage liquid lost systemically as indicated by arrow 48.

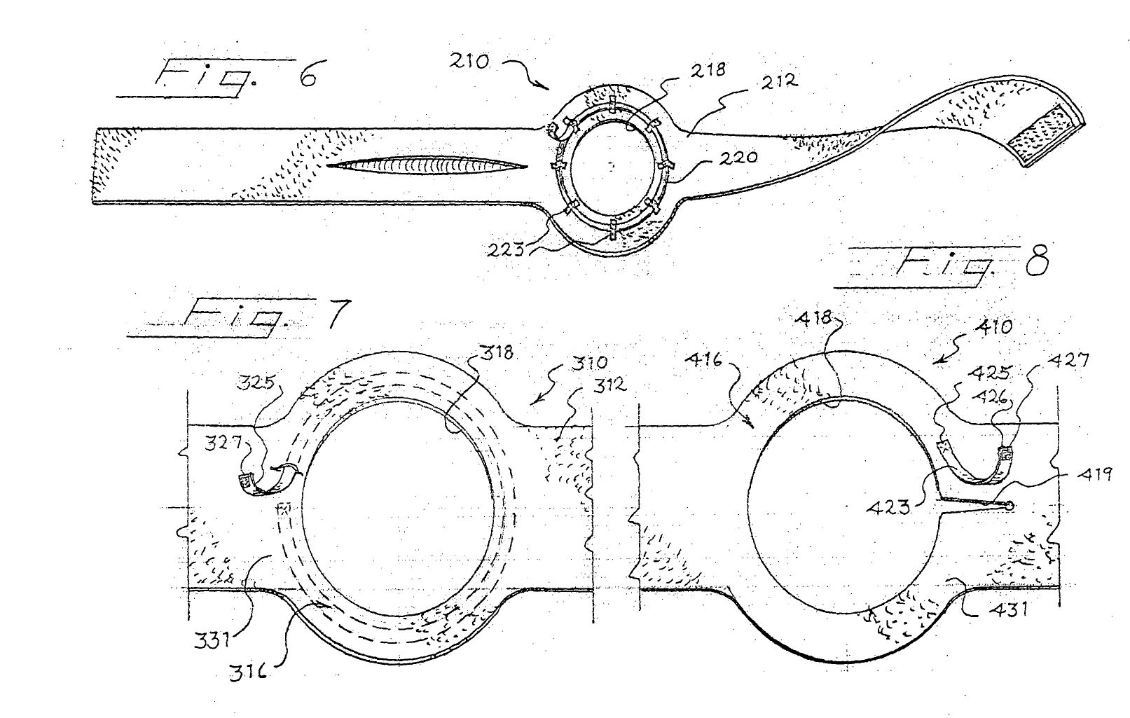

Another embodiment of the present invention is shown in FIG. 5. Harness 110 is provided and is similar to the embodiment discussed with respect to FIG. 1. In this embodiment, however, harness 110 comprises a base member 112 having a first surface 114 and a second surface 116 and including two through apertures 118 and 119. Apertures 118 and 119 are of different diameters so as to accommodate different size breasts. Each of apertures 118 and 119 also include a breast stabilizer 120 and 121 about their respective aperture. In this example, there are only two apertures, however, it is possible for there to be additional apertures. It is further contemplated that the breast stabilizers 120 and 121 may exert different amount of compressive force. For example, breast stabilizer 120 formed about aperture 118, which is larger in diameter than aperture 119 to accommodate larger breasts, may provide more compressive force so as to provide the desired tourniquet effect. Also, breast stabilizer 120 may be wider or more resilient than breast stabilizer 121 in order to enhance the positioning and stabilization of the larger breast, which may have a greater tendency to sag or roll.

Another alternate embodiment of the present invention is shown in FIG. 6. In this embodiment, harness 210 again includes a base member 212 and an aperture 218. The breast stabilizer here includes a clamp, such as adjustable belt 220. Belt 220 may be of varying widths depending on the desired support to be provided by the belt. For example, the belt may be a drawstring. Alternatively, belt 220 may be a wider strap as shown. In this particular embodiment, belt 220 is not integrally formed with the base member 212, but instead, is removable. Belt 220 may be positioned by any suitable means, such as a series of loops 223 about the aperture 218. Alternatively, as shown in FIG. 7, a harness 310 can include a belt 320 that is integrally formed with base member 312 about aperture 318. For example, belt 320 may be passed between layers for base member 312 with one end portion secured therein (not shown) and an end portion 325 extending therefrom, end portion 325 having flexible hooks 327 formed on the end portion 325 which are engageable with the web of entangled fibers 331 on the second surface 316 of base member 312.

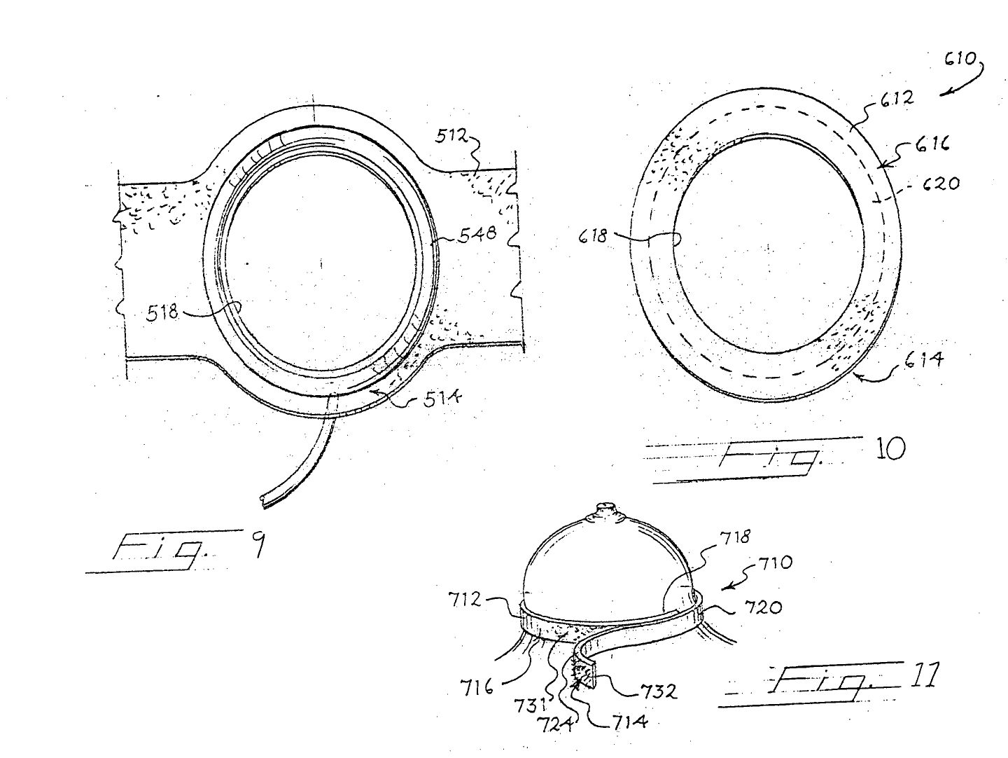

Another embodiment of a harness 410 having a clamp is shown in FIG. 8. In this particular embodiment, aperture 418 includes one or more slits, such as slit 419 extending from the aperture 418. Secured to one side of slit 419 is a first end portion 425 of tab 423. The second end portion 426 of tab 423 comprises a plurality of flexible hooks 427 suitable for engaging the web of entangled fibers 431 on the second surface 416 of base member 412. Slit 419 may be closed by engaging the second end portion 426 of tab 423 with the fibers 431 on the second surface 416 of base member 412, and thereby further compresses the base of the breast.

Yet another embodiment of a harness 510 that includes a component for adjusting the compression of the breast is shown in FIG. 9. In this embodiment an inflatable bead or bladder 548 is provided about aperture 518 on the first side 514 of the base member 512. The pump (not shown), such as a compressible bulb, for inflating the bladder 548 can be an external component or an integral component, as desired. The compressive force exerted on the base of the breast may be adjusted by inflating or deflating bladder 548.

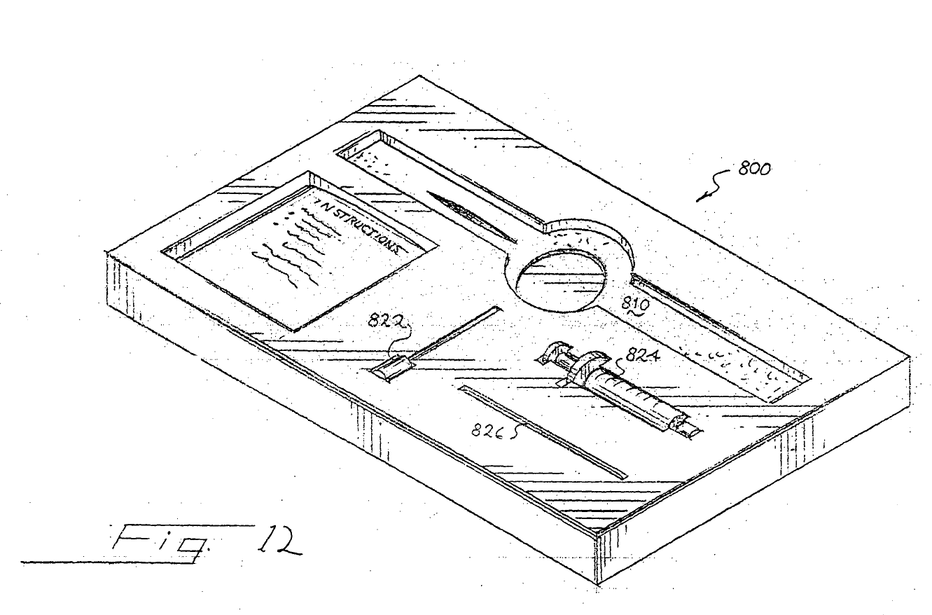

Thus far the embodiments shown have included a torso band. However, such a torso band is not always necessary. A harness 610 such as that shown in FIG. 10 may be utilized to practice the present invention. The harness 610 comprises a base member 612 having a first surface 614 and a second surface 616 and a through aperture 618 sized to receive a human breast therethrough. Located circumferentially about the aperture 618 is provided a breast stabilizer 620. In this embodiment, the breast is guided through aperture 618 in a manner similar to that discussed above. In large breasted women, the breast mass and malleability facilitate entrapping the larger mass without need for securement with a torso band. In order to enhance the supporting of the breast, an optional pressure adhesive layer (not shown) may be included on the first surface 614, such that the harness 610 may be adhesively, yet removably, secured to the patient.

An adjustable collar or belt, as shown in FIG. 11 can also be utilized to practice the method of the present invention. In this embodiment, harness 710 again comprises a base member 712 and breast stabilizer 720. Breast stabilizer 720 is band shaped and is wrapped upon itself to encompass the patient's breast.

Another aspect of the present invention is a packaged kit 800 containing at least one embodiment of the harness 810 of the present invention and instructional indicia 820 as shown in FIG. 11. The kit may also include ductal lavage related devices, such as catheters 822, syringes 824, nipple orifice dilators 826, as well as massage aids. The instructional indicia can include a description of the device, instructions and schematics for using the harness in a clinical procedure, and instructions for assembly of the harness, if applicable. The instructional material can be provided in the form of a label, a printed pamphlet, a video tape, a DVD, a CDROM, a DVDROM, or a combination thereof, and the like.

The foregoing description is to be taken as illustrative, but not limiting. Still other variants within the spirit and scope of the present invention will readily present themselves to those skilled in the art.

1. A harness suitable for reducing systemic losses in ductal lavage procedures, the harness comprising:
a base member having a first surface and a second surface and a through aperture sized to receive a human breast therethrough; and
a breast stabilizer integral with the base member and surrounding the through aperture for radially compressing at least a portion of the breast received in the through aperture.

2. The harness of claim 1, wherein the base member comprises a torso band suitable for enveloping and securement about a torso of a human female patient, the torso band having a first end portion and a second end portion.

3. The harness of claim 2, wherein the first end portion of the torso band is engageable with the second end portion of the torso band.

4. The harness of claim 3, wherein the first end portion of the torso band is engageable with the second end portion of the torso band via engagement between the first surface and the second surface.

5. The harness of claim 4, wherein at least a portion of the second surface includes a web of entangled fibers and at least a portion of the first surface includes a plurality of flexible hooks, the plurality of flexible hooks being engageable with the web of entangled fibers.

6. The harness of claim 1, wherein the breast stabilizer is unitary with the base member.

7. The harness of claim 1, wherein the breast stabilizer is made from an elastomeric material.

8. The harness of claim 1, wherein the circumference of the breast stabilizer is adjustable.

9. The harness of claim 1, wherein the human breast defines a base diameter and the through aperture is of a lesser diameter than the base diameter.

10. The harness of claim 1, wherein the base member further includes an elongated slit spaced from the through aperture.

11. The harness of claim 10, wherein the base member further includes a cover over the elongate slit.

12. The harness of claim 1, wherein the breast stabilizer further includes an adjustable circumferential clamp.

13. The harness of claim 12, wherein the clamp comprises an adjustable belt.

14. The harness of claim 12, wherein the clamp includes adjustable tabs.

15. The harness of claim 12, wherein the clamp mechanism comprises an inflatable bladder.

16. The harness of claim 12, wherein the clamp mechanism is manually adjustable.

17. The harness of claim 1, wherein the base member defines a plurality of apertures, the plurality of apertures being of different diameters.

18. The harness of claim 1, wherein the base member second surface includes a liner disposed thereon.

19. The harness of claim 18, wherein the liner is removable.

20. The harness of claim 18, wherein the liner is replaceable.

21. A harness comprising:
a base member having a first surface and a second surface and a through aperture sized to receive a human breast therethrough;
a breast stabilizer about the through aperture for radially compressing at least a portion of the breast sufficient to reduce systemic losses of lavage liquid during a ductal lavage procedure.

22. The harness of claim 21, where the base member comprises a torso band suitable for enveloping and securement about a torso of a human female patient, the torso band having a first end portion and a second end portion.

23. The harness of claim 22, wherein the first end portion of the torso band is engageable with the second end portion of the torso band.

24. The harness of claim 23, wherein the first end portion of the torso band is engageable with the second end portion of the torso band via engagement between the first surface and the second surface.

25. The harness of claim 24, wherein at least a portion of the second surface includes a web of entangled fibers and at least a portion of the first surface includes a plurality of flexible hooks, the plurality of flexible hooks being engageable with the web of entangled fibers.

26. The harness of claim 21, wherein the breast stabilizer is integral with the base member.

27. The harness of claim 26, wherein the breast stabilizer is unitary with the base member.

28. The harness of claim 21, wherein the breast stabilizer is made of an elastomeric material.

29. The harness of claim 21, wherein the breast stabilizer is adjustable.

30. The harness of claim 21, wherein the human breast defines a base diameter and the through aperture is of a lesser diameter than the base diameter.

31. The harness of claim 21, wherein the base member further includes an elongated slit spaced from the through aperture.

32. The harness of claim 31, wherein the base member further includes a cover over the elongate slit.

33. The harness of claim 21, wherein the breast stabilizer further includes an adjustable circumferential clamp.

34. The harness of claim 33, wherein the clamp is provided with an adjustable belt.

35. The harness of claim 33, wherein the clamp includes adjustable tabs.

36. The harness of claim 33, wherein the clamp is an inflatable bladder circumscribing the through aperture.

37. The harness of claim 33, wherein the clamp is manually adjustable.

38. The harness of claim 21, wherein the base member defines a plurality of apertures having different diameters.

39. The harness of claim 21, wherein the base member second surface includes a liner disposed thereon.

40. The harness of claim 39, wherein the liner is removable.

41. The harness of claim 39, wherein the liner is replaceable.

42. A kit for reducing system losses of ravage liquid during ductal lavage procedures, the kit comprising:
a harness comprising a base member having a first surface and a second surface, the base member defining a through aperture sized to receive a human breast therethrough, and the through aperture being at least partly circumscribed by a breast stabilizer for radially compressing at least a portion of the breast; and
instructional indicia.

43. The kit of 42, further including a catheter, a syringe, and a nipple orifice dilator.

44. A method for reducing systemic losses of lavage liquid during ductal lavage procedures utilizing a harness comprising a base member having a first surface and a second surface, the base member defining a through aperture sized to receive a human breast therethrough, and the through aperture being at least partly circumscribed by a breast stabilizer, the method comprising the steps of:
receiving the human breast through the through aperture;
radially compressing at least a portion of the breast with the breast stabilizer; and
performing a ductal lavage procedure.

45. The method of claim 44 further comprising adjusting the compression on the portion of the breast.

46. The method of claim 44, wherein the step of performing a ductal lavage procedure comprises introducing a rinsing solution into a mammary ducts, and recovering at least a portion of the solution from the mammary ducts.

47. A method for reducing systemic losses of lavage liquid during ductal lavage procedures, the method comprising the steps of:
applying circumferential pressure to a human breast about a base of the breast;
introducing the lavage liquid to a mammary duct in the breast; and
collecting at least a portion of the lavage liquid from the breast.

48. The method of claim 48, wherein the step of applying circumferential pressure to the human breast about the base of the breast is conducted utilizing a harness comprising a base member having a first surface and a second surface, the base member defining a through aperture sized to receive a human breast therethrough, and the through aperture being at least partly circumscribed by a breast stabilizer.
